(19) United States
(12) Patent Application Publication
(10) Pub. No.: US 2012/0095396 A1
(43) Pub. Date: Apr. 19, 2012

(54) Drug Eluting Medical Device Utilizing Bioadhesives

(75) Inventors: Rajesh Radhakrishnan, Maple Grove, MN (US); Steve Larsen, Lino Lakes, MN (US); Scott Schewe, Eden Prairie, MN (US); James Feng, Maple Grove, MN (US); Robert Warner, Woodbury, MN (US); Martyn Folan, Loughrea (IE); Aiden Flanagan, Kilcolgan (IE); John Clarke, Claregalway (IE); Tim O'Connor, Claregalway (IE); Anthony Malone, Miltown (IE)

(73) Assignee: BOSTON SCIENTIFIC SCIMED, INC., Maple Grove, MN (US)

(21) Appl. No.: 13/210,790

(22) Filed: Aug. 16, 2011

Related U.S. Application Data
(60) Provisional application No. 61/394,104, filed on Oct. 18, 2010.

(51) Int. Cl. A61L 29/16; B05D 1/28; B05D 1/02; B05D 5/00; B05D 7/00

(52) U.S. Cl. 604/103.02; 427/2.24

(57) ABSTRACT

A medical balloon having an inner surface and an outer surface comprising a first coating composition comprising at least one therapeutic agent, the first coating composition disposed on the balloon outer surface and forming an interface between the balloon outer surface and the first coating composition and a second coating composition comprising a bioadhesive, the second coating composition disposed on the first coating composition so as to not affect the interface between the balloon outer surface and the first coating composition, the bioadhesive selected so as to adhere to body tissue, and methods of making the same.

### CROSS-REFERENCE TO RELATED APPLICATIONS

This application is claims priority to U.S. Patent Provisional Application No. 61/394,104 filed Oct. 18, 2010, the entire contents of which are hereby incorporated herein by reference.

### FIELD OF THE INVENTION

The present disclosure relates to insertable or implantable medical devices, particularly those employed for drug delivery.

### BACKGROUND OF THE INVENTION

Therapeutic agents are commonly employed during a variety of interventional medical procedures such as PCI (percutaneous coronary intervention) or PTCA (percutaneous transluminal coronary angioplasty), PTRA (percutaneous transluminal renal angioplasty) and POBA (plain old balloon angioplasty), as well as interventional procedures employed in parts of the body other than the cardiovasculature.

It is often desirable that the therapeutic agent be retained at the treatment site for a period of time for the most effective treatment. Therapeutic agents can be rapidly depleted from the treatment site by the constant exposure to bodily fluids.

Medical devices with bioadhesive properties have been employed to treat wounds and deliver therapeutic agents to body tissues. Bioadhesion refers to the ability of certain materials such as, polymers, macromolecules and hydrocolloids to adhere to biological or body tissue. In the past, bioadhesive materials have commonly been used in dentistry, orthopedics, ophthalmology, and in surgical applications. Recently, bioadhesive materials have been used in other areas such as soft tissue-based artificial replacements, and even more recently for controlled release of therapeutic agents to delivery sites. See for example, copending U.S. Patent Application No. 2009/0098176 A1 commonly assigned to Boston Scientific Scimed, Inc. wherein stents and patches are employed for delivery of a therapeutic agent.

### SUMMARY OF THE INVENTION

The present disclosure relates to insertable or implantable medical devices including at least one first coating composition disposed on the surface thereof and at least one second coating composition disposed on the first coating composition. The first coating composition contains a biologically active material and the second coating composition contains a polymeric bioadhesive material.

In one aspect, embodiments of the present disclosure relate to a medical device having an inner surface and an outer surface and including on at least a portion of the outer surface, a first coating composition including at least one therapeutic agent, the first coating composition disposed on the balloon outer surface and forming an interface between the balloon outer surface and the first coating composition and a second coating composition of a bioadhesive, the second coating composition disposed on the first coating composition so as to have no affect on the interface between the balloon outer surface and the first coating composition, where the bioadhesive is selected so as to adhere to body tissue.

In some embodiments the medical device is a balloon.

In some embodiments, the first coating composition is deposited in discrete pattern formations and the second coating composition is deposited precisely on the first coating composition.

In another aspect, the present disclosure relates to a method of applying a coating to a medical balloon in a discrete pattern, the method including providing a medical device having an inner surface and an outer surface, applying at least one therapeutic agent to said outer surface of said medical device in a discrete pattern and applying a bioadhesive over said at least one therapeutic agent.

These and other aspects, embodiments and advantages of the present disclosure will become immediately apparent to those of ordinary skill in the art upon review of the Detailed Description and Claims to follow.

### BRIEF DESCRIPTION OF THE DRAWINGS

FIG. 1 is a perspective view of an embodiment of a balloon having deposits of therapeutic agent deposited in a discrete dot array pattern according to the present disclosure.

FIG. 2 is a cross-section taken at 2-2 in FIG. 1 showing an embodiment of a balloon wall having therapeutic agent and bioadhesive deposited thereon according to the present disclosure.

FIG. 3 is a cross-section taken at 2-2 in FIG. 1 showing an embodiment of a balloon wall having a therapeutic agent, an endothelial cell stimulant and a bioadhesive deposited thereon.

FIG. 4 is a partial view of a balloon wall having therapeutic agent, an endothelial cell stimulant and bioadhesive deposited thereon.

FIG. 5 is a partial view of an embodiment of a balloon wall having therapeutic agent, bioadhesive and a protective coating deposited thereon according to the present disclosure.

FIG. 6 is a perspective view of an embodiment of a balloon wherein wings have been formed therein, the therapeutic agent and bioadhesive are located between the wings.

FIG. 7 is a radial cross-section of a balloon similar to that shown in FIG. 6.

FIG. 8 is a radial cross-section of a balloon similar to that shown in FIG. 6 wherein the wings have been folded about the longitudinal axis of the balloon.

FIG. 9 is a radial cross-section of a balloon similar to that shown in FIG. 6 wherein the balloon has been expanded at a treatment site within a body lumen.

FIG. 10 is a micrograph of an embodiment of a partial balloon surface having therapeutic agent deposited thereon according to the present disclosure.

FIG. 11 is a micrograph of an embodiment of a balloon surface as shown in FIG. 5 in an enlarged view according to the present disclosure.

### DETAILED DESCRIPTION

While embodiments of the present disclosure may take many forms, there are described in detail herein specific embodiments of the present disclosure. This description is an exemplification of the principles of the present disclosure and is not intended to limit the disclosure to the particular embodiments illustrated.

Turning now to the figures, FIG. 1 illustrates one embodiment of a balloon 10 having a first coating composition 20 including at least one therapeutic agent deposited thereon in a discrete pattern and having a second coating composition 22 including a bioadhesive deposited thereon. In this embodiment, the pattern of first coating composition 20 and second coating composition 22 is shown on balloon body 12 only. Although this is one example of a deposition pattern, other patterns may be employed and such deposition does not limit the scope of the present disclosure. The cones 14 and waist 16 could also include deposition of first coating composition 20 and second coating composition 22.

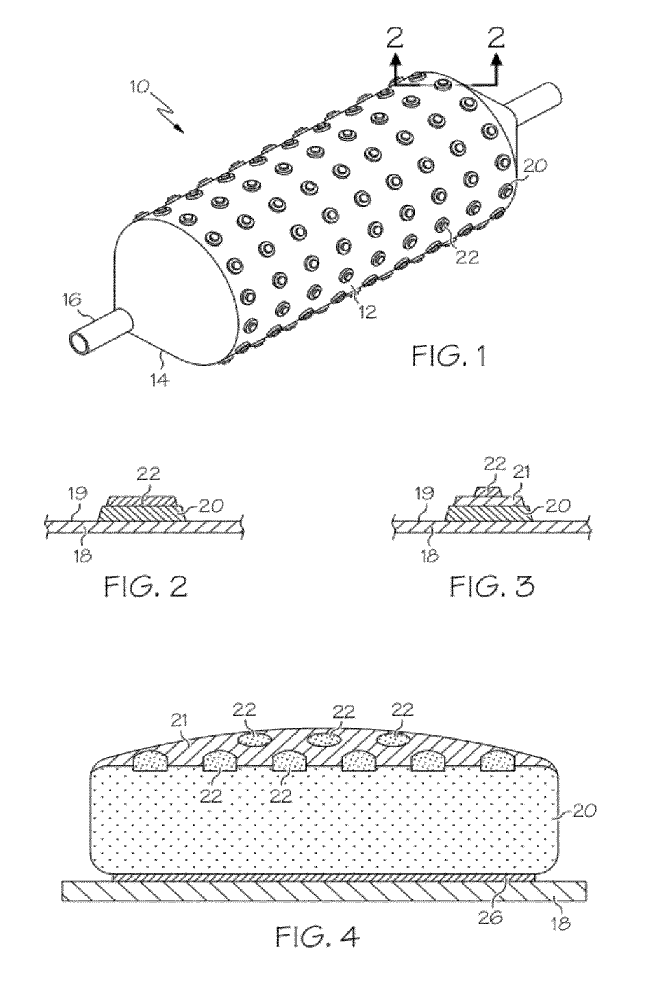

FIG. 2 is a cross-section taken at 2-2 in FIG. 1 showing the outer surface 19 of the balloon wall 18 having a first coating composition 20 including at least one therapeutic agent and a second coating composition 22 including a bioadhesive deposited in a discrete pattern thereon.

FIG. 3 is a cross-section of an alternative embodiment wherein the balloon wall 18 has a first coating composition 20 including at least one therapeutic agent and a second coating composition 22 including a bioadhesive and a third coating composition disposed therebetween. The third coating composition includes an endothelial cell stimulant for promoting increased and/or more rapid uptake of the therapeutic agent by the endothelial cell lining. Suitably, the third coating composition is exposed to the vessel wall.

Alternatively, the endothelial cell stimulant may be included in the first coating composition, the second coating composition or both.

FIG. 4 illustrates another embodiment of the invention wherein the first coating composition 20 including the therapeutic agent is disposed on the balloon wall 18. In this embodiment, a primer composition 26 including a balloon adhesive is shown disposed on the balloon wall 18 prior to deposition of the first coating composition 20. Second coating composition 22 including the bioadhesive is disposed in a discrete pattern on the first coating composition 20. In this embodiment, a third coating composition 21 including the endothelial cell stimulant is disposed between the first coating composition 20 and the second coating composition 22. This illustrates an alternative deposition pattern for the first coating composition 20 and the second coating composition 22.

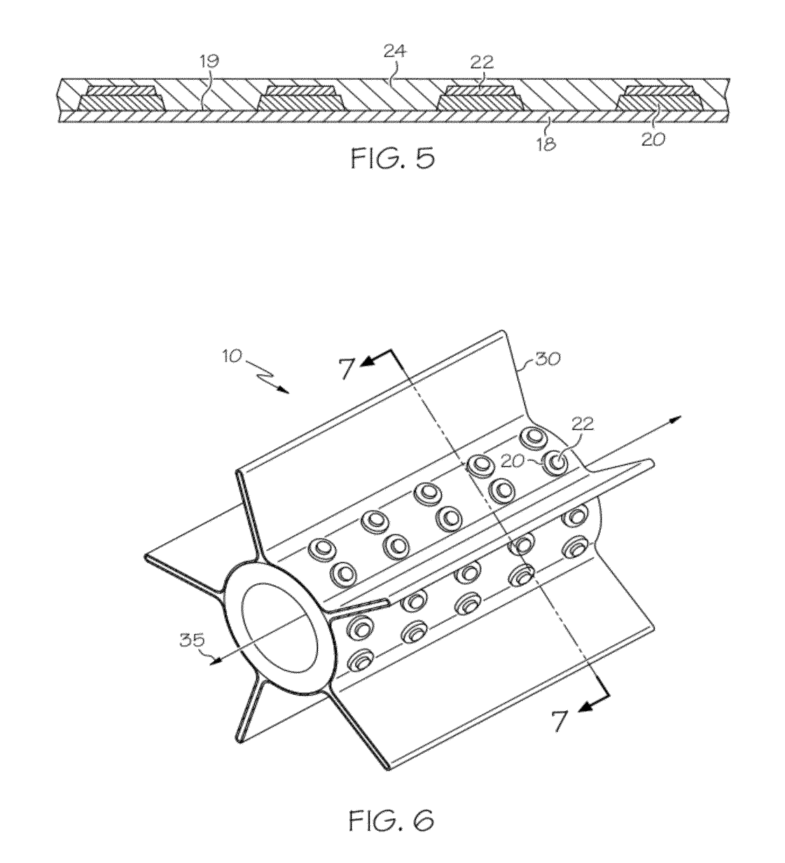

FIG. 5 is an alternative embodiment wherein a protective coating composition 24 is deposited over the first coating composition 20 including at least one therapeutic agent and the second coating composition 22 including the bioadhesive. Suitably, the protective coating composition 24 can be sufficiently gone by the time the medical device is deployed at a treatment site to allow the second coating composition 22 with the bioadhesive to adhere to the body tissue at the treatment site, but remain long enough so that the second coating composition 22 does not prematurely adhere to body tissue during delivery of the device through a body lumen. For example, in some embodiments, the protective coating composition 24 can include a material that is soluble or dispersible in body fluids so as to rapidly dissolve or disperse by the time the medical device is deployed at the treatment site. The protective coating composition 24 can protect the drug from premature release in the body upon exposure to bodily fluids during delivery of the device through a body lumen to the treatment site. Suitably, the protective coating composition 24 can dissolve or disperse, or otherwise allow the second coating composition 22 including the bioadhesive to function at the site of the medical device deployment so that the second coating composition 22 including the bioadhesive can adhere to the vessel wall or tissue at the treatment site but does not prematurely adhere to any tissue or vessel wall during delivery through the body lumen.

For many procedures, the protective coating composition 24 can be sufficiently gone within about 2 minutes to about 15 minutes, in some embodiments, within about 5 minutes to about 10 minutes.

FIG. 6 illustrates an embodiment having an alternative discrete coating pattern. In this embodiment, first coating composition 20 and second coating composition 22 are disposed on the balloon 10 such that when wings 30 are formed in the balloon 10, first coating composition 20 and second coating composition 22 are located in between the wings 30.

Wings 30 can be formed in the balloon 10 using any method known in the art including the use of impinging members while the balloon 10 is being deflated.

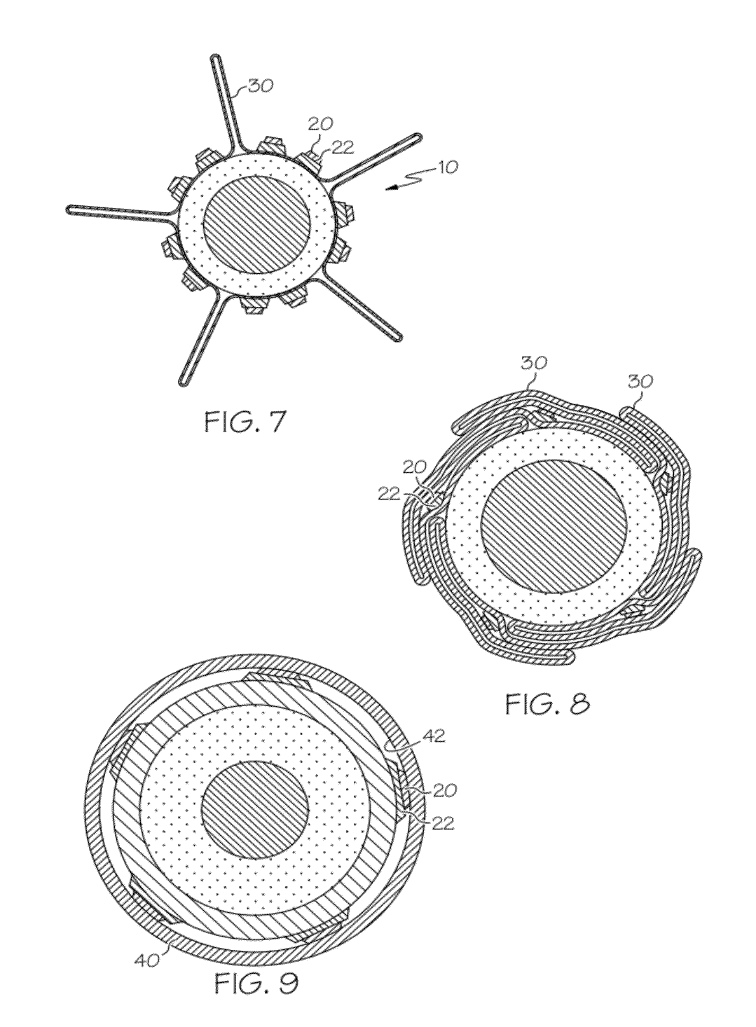

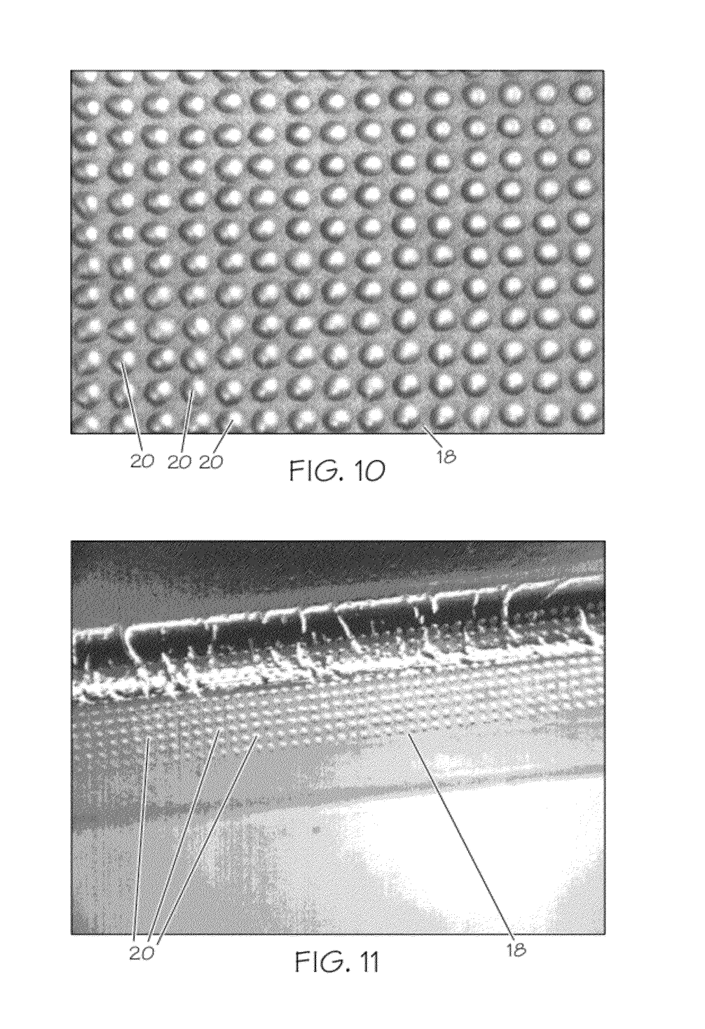

FIG. 7 is a radial cross-section of the balloon in FIG. 6 taken at section 7-7.

FIG. 8 is a cross-sectional view of a deflated balloon 10 as in FIGS. 6 and 7 wherein the wings 30 have been folded or wrapped about the longitudinal axis 35 of the balloon 10.

This embodiment provides benefits in that the wings are shown wrapped about and covering the first 20 and second 22 coating compositions so as to protect them from premature contact with the vessel wall. In this embodiment it may be further beneficial to incorporate a protective coating 24 over the first 20 and second 22 coating compositions as previously discussed with respect to FIG. 5 above. The protective coating will be discussed in more detail below.

FIG. 9 is a cross-sectional view of a balloon as in FIGS. 6-8 wherein the balloon 10 is shown in an expanded state at the treatment site within a vessel 40 of a patient. As can be seen from FIG. 9, the second coating composition 22 including the bioadhesive disposed over the first coating composition 20 is in contact with the vessel wall 42. Once in position at the treatment site, the balloon will be deflated and the wings 30 rewrapped for removal from the vessel 40 leaving behind the second coating composition 22 including the bioadhesive and the first coating composition including the therapeutic agents.

As previously discussed a third coating composition 21 including an endothelial cell stimulant may also be disposed between the first coating composition 20 and the second coating composition 22, or alternatively, the endothelial cell stimulant can be included in the first coating composition 20, the second coating composition 22 or both.

The discrete pattern of the first coating composition 20 including at least one therapeutic agent shown in the above-referenced figures as well as the second coating composition 22 including the bioadhesive can be formed on the balloon surface employing a variety of techniques, one of which is a direct writing technique.

In some embodiments, the adhesion of the first coating composition 20 including the therapeutic agent to the medical device may be weaker than that of the second coating composition 22 including the bioadhesive to the vessel wall or tissue and also, the adhesion of the first coating composition 20 to the medical device may be weaker than that of the first coating composition 20 with the therapeutic agent to the second coating composition 22 including the bioadhesive so that the first coating composition 20 with the therapeutic agent remains with the second coating composition 22 including the bioadhesive at the treatment site.

Suitable therapeutic agents, bioadhesives and protective coating materials are discussed in detail below. A variety of each can be employed herein. The following lists are intended to be illustrative and not exhaustive. Those of ordinary skill in the art will be versed in other materials that could be employed herein.

### Balloon Materials

Polymeric compositions suitable for balloon formation can be employed herein. These materials include non-compliant, semi-compliant and compliant balloon polymer materials.

Examples of suitable balloon materials include, but are not limited to, PET (polyethylene terephthalate) polyethylene, polyvinyl chloride, Surlyn® polyethylene ionomer copolymer, polyurethanes, nylon 12, Pebax® (polyether-block-amide), polyamide-polyether-polyester block copolymer, and polyester-polyether block copolymers. See commonly assigned U.S. Pat. Nos. 6,863,861, 4,490,421, 5,264,260, 4,906,244, 5,328,468, 4,950,239, 5,500,180, 5,556,383, 6,146,356, 6,270,522, 5,344,400, 5,833,657, 5,250,069, 5,797,877 and 5,270,086, each of which is incorporated by reference herein. Methods of forming the balloons are also disclosed therein and include the steps of extruding polymer tubing and radially expanding the tubing in a balloon mold.

### Therapeutic Agents

Various therapeutic agents may be employed herein depending on the condition which is being treated. As used herein, the terms, “therapeutic agent”, “drug”, “pharmaceutically active agent”, “pharmaceutically active material”, “beneficial agent”, “bioactive agent”, and other related terms may be used interchangeably herein and include genetic therapeutic agents, non-genetic therapeutic agents and cells. A drug may be used singly or in combination with other drugs. Drugs include genetic materials, non-genetic materials, and cells.

A therapeutic agent may be a drug or other pharmaceutical product such as non-genetic agents, genetic agents, cellular material, etc. Some examples of suitable non-genetic therapeutic agents include but are not limited to: antithrombogenic agents such as heparin, heparin derivatives, vascular cell growth promoters, growth factor inhibitors, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof.

Other active agents include, but are not limited to, antineoplastic, antiproliferative, antimitotic, antiinflammatory, antiplatelet, anticoagulant, antifibrin, antiproliferative, antibiotic, antioxidant, and antiallergic substances as well as combinations thereof.

Examples of antineoplastic/antiproliferative/antimitotic agents include, but are not limited to, paclitaxel (e.g., TAXOL® by Bristol-Myers Squibb Co., Stamford, Conn.), the olimus family of drugs including sirolimus (rapamycin), biolimus (derivative of sirolimus), everolimus (derivative of sirolimus), zotarolimus (derivative of sirolimus) and tacrolimus, methotrexate, azathiprine, vincristine, vinblastine, 5-fluorouracil, doxorubicin hydrochloride, mitomycin, cisplatin, vinblastine, vincristine, epothilones, endostatin, angiostatin and thymidine kinase inhibitors.

While the preventative and treatment properties of the foregoing therapeutic substances or agents are well-known to those of ordinary skill in the art, the substances or agents are provided by way of example and are not meant to be limiting. Other therapeutic substances are equally applicable for use with the disclosed methods and compositions. See commonly assigned U.S. Patent Application Nos. 2010/0087783, 2010/0069838, 2008/0071358 and 2008/0071350, each of which is incorporated by reference herein. See also commonly assigned U.S. Patent Application Nos. 2004/0215169 and 2009/0098176, and U.S. Pat. No. 6,805,898, each of which is incorporated by reference herein.

Derivatives of many of the above mentioned compounds also exist which are employed as therapeutic agents and of course mixtures of therapeutic agents may also be employed.

For application, the therapeutic agent can be dissolved in a solvent or a cosolvent blend, and an excipient may also be added to the first coating composition.

Suitable solvents include, but are not limited to, dimethyl formamide (DMF), butyl acetate, ethyl acetate, tetrahydrofuran (THF), dichloromethane (DCM), acetone, acetonitrile, dimethyl sulfoxide (DMSO), butyl acetate, etc.

Suitable excipients include, but are not limited to, acetyl tri-n-butyl citrate (ATBC), acetyl triethyl citrate (ATEC), dimethyl tartarate (D, L, DL), diethyl tartarate (D, L, DL), dibutyl tartarate (D, L, DL), mono-, di- and tri-glycerol such as glycerol triacetate (triacetin), glycerol tributyrate (tributyrin), glycerol tricaprylate (tricarprin), sucrose octa acetate, glucose penta acetate (D, L, DL, and other C6 sugar variations), diethyl oxylate, diethyl malonate, diethyl maleate, diethyl succinate, dimethyl glutarate, diethyl glutarate, diethyl 3-hydroxy glutarate, ethyl gluconate (D, L, DL, and other C6 sugar variations), diethyl carbonate, ethylene carbonate, methyl acetoacetate, ethyl acetoacetate, butyl acetoacetate, methyl lactate, (D, L, or DL), dthyl lactate, (D, L, or DL), butyl lactate (D, L, or DL), methyl glycolate, ethyl glycolate, butyl glycolate, lactide (DD), lactide (LL), lactide (DL), glycolide, etc.

Suitable biodegradable polymeric excipients may include polylactide, polylactide-co-glycolide, polycaprolactone, etc.

Other suitable polymeric excipients include, but are not limited to, block copolymers including styrenic block copolymers such as polystyrene-polyisobutylene-polystyrene triblock copolymer (SIBS), hydrogels such as polyethylene oxide, silicone rubber and/or any other suitable polymer material.

These lists are intended for illustrative purposes only, and not as a limitation on the scope of the present disclosure.

### Bioadhesive Materials

Any suitable bioadhesive material may be employed herein and can include natural polymeric materials, as well as synthetic materials, and synthetic materials formed from biological monomers such as sugars. Bioadhesives can also be obtained from the secretions of microbes or by marine molluscs and crustaceans. Bioadhesives are designed to adhere to biological tissue.

The bioadhesives employed herein can have better adhesion to body tissue, and the bioadhesive can have better adhesion to the therapeutic substance than does the therapeutic substance to the medical device.

In other words, the adhesion at the interface between the therapeutic agent and the medical device is weaker than the adhesion at the interface between either the therapeutic agent and the bioadhesive and the bioadhesive and the body tissue this so that the therapeutic agent remains with the bioadhesive when the medical device is retracted from the body.

Examples of bioadhesives include, but are not limited to, amino adhesives, adhesive surface proteins (MSCRAMMS), adhesively modified biodegradable polymers such as Fatty Ester Modified PLA/PLGA, polymer materials, minigel particles, each discussed in detail below, as well as mixtures thereof.

Suitably, the bioadhesive is dissolved in a solvent or cosolvent blend prior to application. Suitable solvents include, but are not limited to, alcohols including methanol, ethanol and isopropanol, and water.

The following examples of bioadhesives are intended for illustrative purposes only, and not as a limitation on the scope of the present disclosure.

### Amino Acids

Amino acids find use in embodiments of the present disclosure. Amino acids can be both utilized to facilitate release from the delivery vehicle as well as to gain adhesion to the lesion site. Zwitterionic amino acids can be employed either as a layer or as a component within the HA/active agent layer. The zwitterionic amino acid can be oriented so that the hydrophobic side of the zwitterionic amino acid selectively facilitates adhesion to the lipophilic lesion. One example of a useful compound is amino acid 3,4-L-dihydroxyphenylalanine (DOPA), a tyrosine derivative found in high concentrations in the “glue” proteins of mussels.

### Adhesive Surface Proteins

Protein adhesions called MSCRAMMs (microbial surface components recognizing adhesive matrix molecules) can also be employed as a bioadhesive in the second coating composition. MSCRAMMS are naturally produced by pathogens to initiate adhesion to the host extra cellular matrix to initiate infection. These adhesive surface proteins can be isolated or synthesized and utilized either as a separate layer or in the HA/active agent composition to facilitate adhesion the lesion site.

### Adhesively Modified Biodegradable Polymers

One example of an adhesively modified biodegradable polymer is a DOPA (L-3,4-dihydroxyphenylalanine) modified PLA (polylactic acid) or PLGA poly(lactide-co-glycolide) having the following structure:

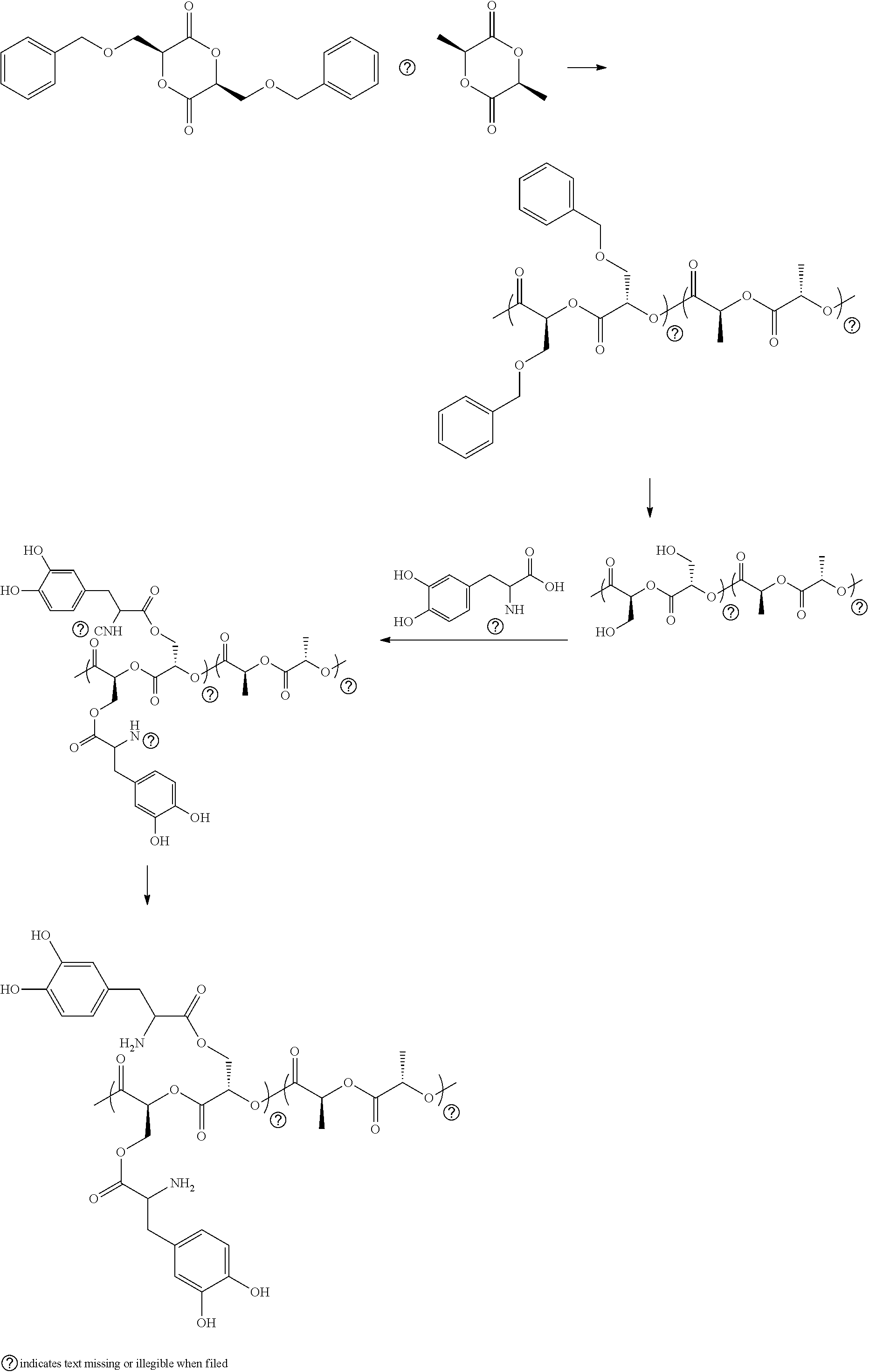

### Fatty Ester Modified PLA/PLGA

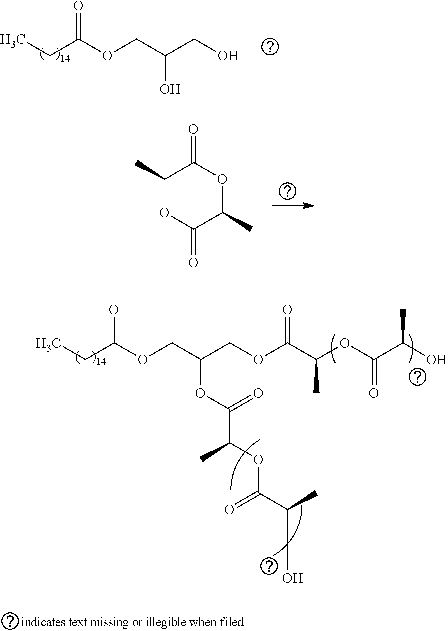

In this embodiment, examples of suitable adhesive moieties include, but are not limited to, monopalmitate (shown above), monostearin, glycerol, aa dilaurin or iso-stearyl alcohol.

### Polymer Materials

Proteins such as gelatin and carbohydrates such as starch may also be employed herein. Polysaccharides such as sorbitol, sucrose, xylitol, anionic hydrated polysaccharides such as gellan, curdlan, XM-6 and xanthan may also be employed as a bioadhesive herein. Others include derivatives of natural compositions such as algenic acid, hydrated gels and the like, and also biocompatable polymers and oligomers such as dextrans, dextranes and dextrins, hydrogels including, but not limited to, polyethylene glycol (PEG), polyethylene glycol/dextran aldehyde, polyethylene oxide, polypropyline oxide, polyvinylpyrrolidine, polyvinyl acetate, polyhydroxyethyl methacrylate and polyvinyl alcohol, as well as derivatives thereof may also be employed herein. See for example U.S. Pat. No. 6,391,033, the entire content of which is incorporated by reference herein.

### Minigel Particles

Another bioadhesive is poly(NIPAM) (poly(N-isopropylacrylamide) minigel particles. This polymer has the property of being in a liquid state at room temperature and an adhesive at body temperature.

See “Preparation and Swelling Properties of Poly(NIPAM) “Minigel” Particles Prepared by Inverse Suspension Polymerization”, Dowding, John et al., Journal of Colloid and Interface Science 221, 268-272 (2000), available online at http:/www.idealibrary.com, the entire content of which is incorporated by reference herein.

For better retention of the polymer on the balloon surface, several techniques may be employed. Suitably, the minigel particles are crosslinked or mixed with a higher molecular weight polymer to allow enough time for retention of the minigel to the medical device during delivery, or uncrosslinked minigel particles can be employed in a crosslinked polymer network.

For example, an uncrosslinked minigel such as poly(N-isopropylacrylamide) may be employed with the reaction product of a vinyl polymer. See commonly assigned U.S. Pat. No. 5,693,034, the entire content of which is incorporated by reference herein.

Poly(N-isopropylacrylamide) may also blended with a higher molecular weight polymer such as a higher molecular weight hydrogel polymer. Examples of hydrogels include, but are not limited to, polyvinylpyrrolidone, polyacrylamides, polyethylene oxide, polyacrylic acid, poly (sodium-4-styrenesulfonate), poly(3-hydroxybutyric acid), and 2-hydroxyethyl methacrylate.

These examples are intended for illustrative purposes only, and not as a limitation on the scope of the present disclosure.

### Endothelial Cell Stimulant

In some embodiments it may be desirable to employ an endothelial cell stimulant either as part of the coating composition comprising the therapeutic agent, or as an additional coating composition deposited between the coating composition comprising the therapeutic agent and the coating composition comprising the bioadhesive.

It is desirable that portions of the coating including the endothelial cell stimulant are exposed to the vasculature and come into contact with the endothelial cells lining the vasculature at the treatment site. Endothelial cell stimulants promote increased and/or more rapid uptake of the therapeutic agent(s).

Examples of materials which can stimulate the endothelial cell lining include, but are not limited to, monosaccharides such as glucose, sorbitol, fructose, galactose, xylose and ribose, disaccharides such as maltose or sucrose and polymers thereof such as dextrins and maltodextrins.

These examples are intended for illustrative purposes only and not as a limitation on the scope of the present invention.

### Protective Coating

In some embodiments, a protective coating composition is employed over both the first coating composition including at least one therapeutic agent and the second coating composition including the bioadhesive. The protective coating composition can provide temporary protection of the second coating composition including the bioadhesive before it reaches the target location so that the bioadhesive does not come in contact with tissue before it reaches the treatment location which could result in a premature loss of the therapeutic agent. The protective coating composition is sufficiently gone by the time the medical device reaches the deployment site so as to allow the second coating composition with the bioadhesive to adhere to the body tissue at the deployment site, for example, when the balloon is expanded. However, the protective coating composition remains long enough so that the second coating composition including the bioadhesive does not prematurely adhere to body tissue during delivery of the device through a body lumen. For example, the protective coating composition rapidly dissolves or disperses in body fluids, or partially dissolves or disperses, or does not otherwise interfere with adhesion between the bioadhesive and the biological tissue. For some procedures, the protective coating composition is sufficiently gone within about 2 to about 15 minutes, and in some embodiments, the protective coating composition is sufficiently gone within about 5 to about 10 minutes.

Examples of suitable materials for use in the protective coating composition include, but are not limited to, salts, sugars and polymers.

Specific examples of suitable materials include, but are not limited to potassium chloride, heparin, mannitol or ReoPro® (abciximab) for example. See U.S. Patent Application Nos. 2003/0060877 and 2007/0078413 each of which is incorporated by reference herein in their entirety.

Specific examples of polymer materials include, but are not limited to, polyvinyl alcohol (PVOH), polyvinyl acetate (PVA), and so forth. Specific PVA polymers may be purchased from Adept Polymers Limited, Unit 7, Woodrow Way, Fairhills Industrial Estate, Irlam, Manchester, M44 6ZQ under the name of Depart Products, W-50 product series.

### Coating Methods

Suitable coating methods may be employed herein including spraying, dipping, brushing, etc. The therapeutic agent and the bioadhesive are applied to at least a portion of the outer surface of the medical device in any pattern desired.

In one embodiment, a balloon is coated with a discrete coating pattern of the first coating composition including at least one therapeutic agent and second coating composition including the bioadhesive using a variety of methods including ink jet technology and or direct write technologies such as the Optomec® aerosol jet technology available from Optomec® located in Albuquerque, N. Mex.

Shown in FIGS. 4 and 5 is a dot array of Paclitaxel applied to the balloon using the Optomec® aerosol jet technology. Paclitaxel and acetyl tri-n-butyl citrate (excipient) at a ratio of 70:30 on a solids basis was applied out of a cosolvent mixture of butyl acetate and dimethyl formamide at a ratio of 10:90. Paclitaxel is commercially available from Angiotech located in Vancouver, BC. Acetyl tri-n-butyl citrate is commercially available from Vertellus Specialties, Inc. located in Greensboro, N.C. The solution contained 10 percent solids resulting in 2 micrograms of Paclitaxel per square millimeter.

The ratios of drug to excipient, the amount of solids employed and the solvent/cosolvent blend employed can be varied. For example, 20% or less solids may be employed and in some cases, it may be desirable to employ less than 10% solids. Butyl acetate and dimethyl formamide can be used alone, or as a cosolvent blend and the ratio in the cosolvent blend can be varied, for example, a ratio of butyl acetate to DMF of 20:80 was also employed, and the ratio of drug to excipient may be 80:20. The deposited drug is suitably 2 micrograms/square millimeter or less.

These are intended to be illustrative examples, and not as a limitation on the scope of the present disclosure.

### Example 1

PolyNIPAM, 10 percent solids, was dissolved in a 50/50 cosolvent blend of methanol and water. The ratio of methanol to water may be varied and either solvent may be employed alone as well. The bioadhesive was applied over the bioadhesive using the same dot array pattern so as to have no effect on the balloon/drug interface. The solvent was allowed to evaporate (heat may be employed to facilitate evaporation) and the poly(NIPAM) was then crosslinked via electron beam (EB) irradiation. Gamma radiation may also be employed. See, for example, Panda et al., “Synthesis and swelling characteristics of poly (N-isopropylacrylamide) temperature sensitive hydrogels crosslinked by electron beam irradiation,” Radiation Physics and Chemistry, 58, (2000), pp. 101-110, the entire content of which is incorporated by reference herein.

FIG. 4 is a micrograph of a partial balloon surface 18 having therapeutic agent 20 deposited thereon and FIG. 5 is an enlarged view of balloon surface 18 having therapeutic agent 20 deposited thereon in a discrete dot array pattern. The same technology can be employed to apply the second coating composition with the bioadhesive using the same pattern precisely over the therapeutic agent 20.

Balloon wall 18 is shown having microdot array of discrete areas of therapeutic agent 20, in this example, Paclitaxel.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference in their entirety into the specification to the same extent as if each individual publication, patent or patent application was specifically and individually indicated to be incorporated herein by reference. Citation or discussion of a reference herein shall not be construed as an admission that such is prior art.

1. A medical balloon, the medical balloon having an inner surface and an outer surface, waist, cone and body portions, the balloon comprising:
a first coating composition comprising at least one therapeutic agent, the first coating composition disposed on the balloon outer surface and forming an interface between the balloon outer surface and the first coating composition; and
a second coating composition comprising a bioadhesive, the second coating composition disposed on the first coating composition, the bioadhesive selected so as to adhere to biological tissue.

2. The medical balloon of claim 1 wherein the second coating composition is disposed on the first coating composition so as to have no affect on the interface between the balloon outer surface and the first coating composition.

3. The medical balloon of claim 1 wherein the first coating composition is applied over at least the balloon portion and the second coating composition is disposed over the first coating composition in a discrete pattern.

4. The medical balloon of claim 1 wherein the first coating composition is disposed on the balloon outer surface in a discrete pattern.

5. The medical balloon of claim 4 wherein the second coating composition is disposed on the first coating composition in said same discrete pattern.

6. The medical balloon of claim 1 further comprising a third coating composition disposed between the first coating composition and the second coating composition, the third coating composition comprising an endothelial cell stimulant.

7. The medical balloon of claim 6 wherein said third coating composition comprises at least one endothelial cell stimulant selected from the group consisting of monosaccharides, disaccharides, polymers thereof and mixtures thereof.

8. The medical balloon of claim 7 wherein said endothelial cell stimulant comprises at least one member selected from the group consisting of glucose, sorbitol, fructose, galactose, xylose, ribose, maltose, sucrose, dextrins, maltodextrins and mixtures thereof.

9. The medical balloon of claim 1 wherein said first coating composition comprises an endothelial cell stimulant.

10. The medical balloon of claim 1 wherein the adhesion at the interface between the therapeutic agent and the balloon outer surface is weaker than the adhesion between the therapeutic agent and the second coating composition comprising the bioadhesive and between the tissue and the second coating composition comprising the bioadhesive.

11. The medical balloon of claim 1 further comprising a protective coating composition disposed on said second coating, the protective coating composition comprising a material which dissolves or disperses in body fluids.

12. The medical balloon of claim 11 wherein the protective coating composition comprises salt or sugar.

13. The medical balloon of claim 1 wherein the first coating composition comprises paclitaxel or everolimus.

14. The medical device of claim 1 wherein the second coating composition comprises amino acids, L-3,4-dihydroxyphenylalanine modified polylactic acid, L-3,4-dihydroxyphenylalanine polylactide-co-glycolide, fatty ester modified polylactic acid, fatty ester modified polylactide-co-glycolide, adhesive surface proteins, polysaccharides, poly(N-isopropylacrylamide), polyethylene glycol/dextrin aldehyde, and mixtures thereof.

15. The medical balloon of claim 1 wherein said bioadhesive is a fatty ester modified polylactic acid or a fatty ester modified poly(lactide-co-glycolide), the fatty ester is a member selected from the group consisting of monopalmitate, monostearin, glycerol, aa dilaurin and iso-stearyl alcohol.

16. The medical balloon of claim 1 wherein said second coating composition comprises a poly(N-isopropylacrylamide) bioadhesive.

17. The medical balloon of claim 1 wherein said first coating composition is disposed on the balloon in a discrete pattern and the second coating composition is disposed precisely on the first coating composition.

18. The medical balloon of claim 17 wherein said discrete pattern is a microdot array pattern.

19. A medical balloon, the medical balloon having an inner surface and an outer surface, the balloon comprising:
a first coating composition comprising at least one therapeutic agent, the first coating composition disposed on the balloon outer surface and forming an interface between the balloon outer surface and the first coating composition; and
a second coating composition comprising a bioadhesive, the second coating disposed on the first coating composition so as to have no affect on the interface between the balloon outer surface and the first coating composition, the bioadhesive selected so as to adhere to biological tissue; and
wherein the adhesion at the interface between the therapeutic agent and the balloon outer surface is weaker than the adhesion between the therapeutic agent and the second coating composition comprising the bioadhesive.

20. A method of coating a medical balloon with a discrete pattern, the method comprising:
providing a medical balloon having an inner and an outer surface;
depositing a first coating composition comprising at least one therapeutic agent to the outer surface of the balloon in a discrete pattern; and
depositing a second coating comprising at least one bioadhesive in the same discrete pattern, wherein the second coating composition is deposited precisely on the first coating composition.

21. The method of claim 20 wherein the first coating composition and second coating composition are deposited on the outer surface of the balloon using a direct writing or an aerosol jet application.

22. The method of claim 20 wherein said first coating composition and said second coating composition are deposited in a dot array pattern.

23. The method of claim 20 further comprising the step of applying a protective coating composition over the first coating composition and second coating composition and over the balloon outer surface.

24. The method of claim 23 wherein said protective coating composition comprises salt or sugar.

25. The method of claim 20 wherein said at least one therapeutic agent comprises at least one member selected from the group consisting of the sirolimus, everolimus, biolimus, tacrolimus, zotarolimus and paclitaxel.

26. The method of claim 20 wherein said at least one bioadhesive comprises a member selected from the group consisting of amino acids, L-3,4-dihydroxyphenylalanine modified polylactic acid, L-3,4-dihydroxyphenylalanine polylactide-co-glycolide, fatty ester modified polylactic acid, fatty ester modified polylactide-co-glycolide, adhesive surface proteins, polysaccharides, poly(N-isopropylacrylamide), polyethylene glycol/dextrin aldehyde, and mixtures thereof.

27. The method of claim 20 wherein said at least one bioadhesive is poly(N-isopropylacrylamide.
